(19) United States
(12) Patent Application Publication
(10) Pub. No.: US 2003/0171698 A1
(43) Pub. Date: Sep. 11, 2003

(54) Conductivity-normalized urinary analyte concentration measurement for use in disease diagnosis

(76) Inventors: Yoram Rubin, Haifa (IL); Shai Nimri, Kibbutz Nir David (IL); Nitsa Galili-Nachshon, Gilboa (IL); Sari Alon, Yuvalim (IL); Inbal Ben-Tzi Tzchori, Yehoshua (IL)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(21) Appl. No.: 10/341,526

(22) Filed: Jan. 13, 2003

Related U.S. Application Data
(63) Continuation of application No. PCT/IL01/00642, filed on Jul. 12, 2001.

(30) Foreign Application Priority Data
Jul. 13, 2000 (IL) 137308

(51) Int. Cl. A61B 5/00

(52) U.S. Cl. 600/584

(57) ABSTRACT

The invention relates to a method for the diagnosis of a medical condition in a subject, comprising the steps of measuring the concentration of one or more analytes, relevant for the diagnosis of said medical condition, in a urine sample; measuring the electrical conductivity of said urine sample; obtaining a normalized value for the analyte concentration by dividing said analyte concentration by the electrical conductivity; and determining whether said subject is suffering from said medical condition by means of comparing the normalized value with a pre-determined reference value. The medical condition to be diagnosed can be, for example, an acute cardiac condition for which the relevant urinary analyte can be one or more thromboxanes.

### RELATED APPLICATION

This application is a continuation of International Application PCT/IL01/00642 filed Jul. 12, 2001 designating the United States of America, for which priority is claimed under 35 USC 120.

### BACKGROUND OF THE INVENTION

1. Field of the Invention

The present invention is concerned with the use of measurements of urinary electrical conductivity and of the concentrations of relevant urinary analytes in disease diagnosis. More particularly, the present invention relates to the use of conductivity-normalized measurements of urinary thromboxane concentrations in the diagnosis of acute cardiac conditions.

2. Prior Art

One of the key requirements for the effective management of medical conditions is the availability of accurate, convenient and rapid methods of diagnosis. While this is true of all disease states, it is perhaps most relevant in the case of potentially life-threatening conditions such as acute cardiac disease.

The group of diseases affecting the heart and blood vessels is one of the leading causes of morbidity and mortality. In particular, Acute Coronary Syndrome (ACS) is a leading cause of death in the Western world. While the group of cardiovascular disease taken as a whole consists of a large number of different disease entities, each with it own specific pathogenetic factors, a common element among many of the most prevalent cardiovascular conditions is the formation of athersclerotic plaque, with all its varied biochemical and pathophysiological consequences.

On a worldwide scale, more than 70 million people present at hospitals and other primary health care providers complaining of chest pain each year. In the United States alone, over six million people present with chest pain each year, a statistic that is reflected in the fact that cardiovascular disease accounts for fully one quarter of the current annual health expenditure in the US.

Since the effectiveness of treatment falls exponentially from the time of a myocardial event, the ability to rapidly and accurately diagnose cardiovascular pathology, and thereby commence appropriate treatment at a much earlier stage, is critical in reducing the number of deaths from heart disease.

An additional medical benefit to be derived from improved diagnostic technology screening is the capability to detect patients at risk of developing atherosclerotic lesions and subsequent cardiovascular (and cerebrovascular) pathology. This is of obvious benefit to the development of reliable strategies for the prevention of serious cardiovascular disease.

Finally, the development of early and accurate diagnostic tests will enable health services to reduce the number of unnecessary hospital stays and expensive tests that are administered, providing significant cost savings. Currently, the total annual cost of testing patients for ACS, according to the American College of Cardiology, is estimated to be about $6 billion.

The thromboxanes are compounds derived from prostaglandin endoperoxides that cause platelet aggregation, arterial contraction and many other biological effects. One such compound, thromboxane A₂, a highly unstable biologically active bicyclic oxitane-oxane compound, displays very potent vasoconstricting and platelet aggregating properties. thromboxane A₂ has been found to play a crucial role in atherothrombotic disorders, and increased synthesis thereof has been found to occur immediately following events such as unstable angina and acute myocardial infarction [Fitzgerald, D. J. et al. (1986) N. Engl. J. Med. 315: 983-989]. As mentioned above, thromboxane A₂ is very unstable, and is rapidly converted to stable metabolites such as 11-dehydrothromboxane B₂ and 2,3, di-northromboxane B₂ (collectively referred to hereinbelow as “thromboxane B₂”), which are excreted in the urine.

The electrical properties of ion-containing solutions may be expressed in several different ways. One such defining property is that of resistance to electrical current. Electrical resistance (R) may be defined as:

R=ρ×(L/A)

wherein L and A are respectively the length and cross-sectional area of the medium whose resistance is being determined, and ρ is the constant of proportionality known as the resistivity of the sample. A commonly used measure of the electrical properties of an electrolytic solution is conductivity, which is defined as the inverse of the resistivity, and is usually measured in units of Siemens per centimeter (S/cm).

The conductivity of urine is, inter alia, a measure of urinary dilution, and as such has been used in investigations of the pathogenesis of several different disease states. One such example is the relationship between the degree of urinary dilution and the risk of calcium oxalate crystallization. In one study [Tiselius, H. G. (1992) J. Urol. 148: 990-994], the electrical conductivity of urine was determined in order to assess the degree of urinary dilution. The results of this study suggested that the monitoring of urine dilution by use of a conductivity meter may provide a useful monitoring tool for the prevention of calcium stone disease.

It is a purpose of this invention to provide a urine analysis method for the reliable diagnosis of a variety of medical and surgical conditions.

It is another purpose of the invention to provide such a method that is technically simple to perform, yielding rapid results, thus obviating the need for long waiting periods before appropriate treatment regimes can be initiated.

It is a particular aim of the present invention to provide such a method that may be used in the diagnosis of acute cardiac conditions.

It is a further object of the present invention to provide a diagnostic assay that may be used very early in the development of cardiac conditions, such that it may be used as an early-warning, first-window technique, yielding reliable predictive data.

It is a further purpose of the invention to provide an assay for the diagnosis of acute cardiac conditions that overcomes the problems of prior art methods.

Other objects and advantages of the invention will become apparent as the description proceeds.

### SUMMARY OF THE INVENTION

It has now been surprisingly found that the measurement of both the electrical conductivity and the concentration of relevant analytes in urine samples may be used as a reliable diagnostic and predictive tool. Unexpectedly, it was found that the use of conductivity determinations as a means for normalizing urinary analyte concentrations leads to a significant increase in diagnostic reliability and accuracy.

The invention is primarily directed to a method for the diagnosis of a medical condition in a subject, comprising the steps of:

a) obtaining a sample of urine from said subject;

b) measuring the concentration of one or more analytes relevant for the diagnosis of said medical condition in said urine sample;

c) measuring the electrical conductivity of said urine sample;

d) obtaining a normalized value (NTV) for the analyte concentration by dividing the analyte concentration obtained in step b) by the electrical conductivity of the urine sample obtained in step c);

e) determining whether said subject is suffering from said medical condition by means of comparing the NTV obtained in step d) with a pre-determined reference value.

The above-mentioned steps b) and c) may be performed in any order, or alternatively, may be performed simultaneously.

The term cardiac condition as used herein is to be taken to mean pathological conditions of the heart or blood vessels, including atherosclerotic conditions and pathological thrombogenic conditions.

In a preferred embodiment of the method of the invention, the medical condition to be diagnosed is an acute cardiac condition, and the relevant urinary analyte comprises one or more thromboxanes selected from the group consisting of thromboxane B₂, 11-dehydrothromboxane B₂, 2,3-di-northromboxane B₂, and mixtures thereof.

In a preferred embodiment of the method of the invention, the thromboxane measured is thromboxane B₂.

Many different assay techniques may be used to perform the measurements required by the method of the invention. In one preferred embodiment of the invention, both the analyte (e.g. thromboxane) concentration and the electrical conductivity are measured using an amperometric assay. A suitable amperometric assay for use in the method of the present invention is disclosed in co-pending Israeli patent application no. 132410.

In another preferred embodiment, both the analyte (e.g. thromboxane) concentration and the electrical conductivity are measured using a semiconductor-based device.

In yet another preferred embodiment of the invention, the electrical conductivity of the urine sample is measured using a conductivity meter.

In a further preferred embodiment of the method of the invention, the analyte (e.g. thromboxane) concentration is determined using an immunoassay. In a more preferred embodiment, said immunoassay is an enzymeimmunoassay (EIA).

All the above and other characteristics and advantages of the invention will be further understood from the following illustrative and non-limitative examples of preferred embodiments thereof.

### DETAILED DESCRIPTION OF PREFERRED EMBODIMENTS

The urinary concentration of the relevant analyte, for example thromboxane B₂ compounds, may be determined by the use of any suitable quantitative or semi-quantitative technique. Such suitable techniques include, but are not limited to, enzyme linked immunoassays (ELISA), radioimmunoassays (RIA), immunoturbidimetric assays, amperometric assays, dipstick-type assays, and measurements using semiconductor-based devices. All of the aforementioned techniques are extensively described in the art, and well known to the skilled artisan.

Urinary electrical conductivity may be measured by any of the standard techniques known in the art, including the use of amperometric techniques and dedicated conductivity meters. Solid-state semiconductor devices may also be used. Most conveniently, both the urinary thromboxane concentration and the electrical conductivity determination could be obtained simultaneously by the use of separate channels of the semiconductor device. The two, separate, electrical currents thus generated could be analyzed separately and diagnostic results could be produced, in accordance with the data analysis technique that is described in the following Example.

### EXAMPLE

### Correlation of Thromboxane/Conductivity Measurements with Clinical Diagnosis

Methods

Subjects and Samples:

A group of 89 patients in the age range 40-70 presenting in the Emergency Room of a large district hospital were randomly selected for this study. Samples of urine were collected from each of the patients before they were subjected to any diagnostic or treatment procedures. These urine samples were immediately frozen and stored at −20° C. for periods of less than one month, prior to being used for the biochemical analyses.

The patients were also asked whether they were currently taking, or had recently been taking, cyclooxygenase inhibitors such as aspirin.

The medical condition of each patient was also assessed 30 days after taking the urine sample, according to the following two criteria:

1. Any cardiac event

2. Free of chest pain

In making the above clinical assessment, the set of patients was divided into the following sub-groups according to the outcome on admission (i.e. on the same day that the urine samples were obtained):

a. Referral to cardiac care unit/cardiology department

b. Referral to hospital internal medicine department

c. Discharged

d. All patients

Comparison of the clinical outcome with the result obtained from the biochemical/conductivity analyses (see below in “Data analysis methods”) was performed, in order to determine the sensitivity and specificity of said analyses as diagnostic tools.

Analytical Methods:

1. Thromboxane B₂ analysis

The concentrations of thromboxane B₂ in the urine samples were measured using a modification of the Biotrak™ system (Amersham International plc, Little Chalfont, Buckinghamshire, England; code RPN 220). The frozen urine samples were thawed and used directly in the thromboxane assay, without any form of pretreatment.

Briefly, 50 μl of each sample or thromboxane B₂ standard was added in duplicate to the wells of a microtitre plate pre-coated with donkey anti-rabbit IgG. All standard solution dilutions were made in an assay buffer consisting of 0.1M phosphate buffer, pH 7.5 containing 0.9% sodium chloride and 0.1% bovine serum albumin. The same buffer was also used in the preparation of the zero standard (i.e. 0 pg thromboxane B₂) and non-specific binding (i.e. buffer-only) wells. The amount of thromboxane B₂ added to the standard wells varied between 0.5 and 64 pg per well. Next, 50 μl of rabbit anti-thromboxane B₂ antiserum was added to each well (except for the spectrophotometric blank well). Following this, 50 μl of thromboxane B₂-horseradish peroxidase conjugate solution was added to each well (except for the blank well), and the plate incubated with shaking at room temperature for one hour. At the end of this incubation period, the contents of each well were aspirated, and each well washed four times with 400 μl wash buffer (0.01M phosphate buffer, pH 7.5, containing 0.05% Tween 20). Immediately following the final washing step, 150 μl of enzyme substrate (consisting of 3,3′, 5,5′-tetramethylbenzidine and hydrogen peroxide) were added to each well. The plate was then incubated with shaking at room temperature for exactly 15 minutes, to allow development of the colored reaction product. The reaction was stopped by the addition of 100 μl of 1M sulphuric acid into each well. Following thorough mixing, and withing 30 minutes of addition of the sulphuric acid, the optical density of each well at 450 nm was determined using a plate reader.

A calibration curve was constructed for the thromboxane B₂ standards by plotting the known thromboxane B amount (x-axis) against the percentage of bound antibody (%B/B₀). The latter parameter was calculated according to the following relationship:

%B/B₀=[(thromboxane standard OD−non-specific binding OD)/(B₀ OD−non-specific binding OD)]×100

(wherein each OD reading is the average for duplicate wells).

The sample thromboxane B₂ amounts for the samples were obtained by reading directly from the calibration curve.

2. Conductivity analysis

The electrical conductivity of each of the urine samples was measured using a CyberScan CON100 conductivity meter (Eutech Instruments Pte Ltd., Singapore).

3. Normalization of results

A normalized thromboxane B₂ concentration value for each sample tested was obtained by dividing said thromboxane concentration (measured in pg/ml) by the conductivity value (measured in mS/cm), either by using simple division or by using a more advanced statistical model. For easier analysis, all values were transformed into their natural logarithms. Thus the normalization of the thromboxane concentrations was achieved by the subtraction of the natural logarithm of the conductivity value from the natural logarithm of the thromboxane concentration.

Data Analysis Methods:

The cut-off indicates a value which dictates if the patient condition is pathological or normal. Cut-off was determined according to Receiver Operating characteristic Curves (ROC), which is a plot of the sensitivity (or the true positive value) vs. the false positive value. This analysis optimizes the correlation between the test results and the clinical outcome.

The results of the various analyses described hereinabove were collected and analyzed according to the following two interpretive ‘rules’.

Rule 1—based on measurement of thromboxane B₂ concentration alone, wherein a positive result (i.e. the presence of cardiac disease) is indicated by a natural logarithm-transformed thromboxane value greater than the cut-off value of 5.1 for patients not taking cyclooxygenase inhibiting drugs (e.g. aspirin), or greater than the cut-off value of 4.9, for patients that are taking or have recently taken such drugs.

Rule 2—based on determination of (ln thromboxane B₂ concentration-ln conductivity value) (i.e. the logarithmic transformation of the ratio of the thromboxane B₂ concentration to the conductivity reading), wherein a positive result (i.e. the presence of cardiac disease) is indicated by a result greater than the cut-off value of 3.2, for patients not taking cyclooxygenase inhibiting drugs (e.g. aspirin), or greater than the cut-off value of 2.7, for patients that are taking or have recently taken such drugs.

The cut-off values are the reference values used in the method of the invention. Preferably, such reference values are based on results of diagnostic tests of large groups of patients.

Following analysis of the data according to the above rules, and tabulation of said data, the sensitivity and specificity of each rule was determined according to the following definitions:

Sensitivity (%)=True positive/(False negative+True positive)×100

Specificity (%)=True negative/(False positive+True negative)×100

Results:

The results comparing the clinical outcome (any cardiac event/free of chest pain) with the laboratory results, as interpreted by each of the two aforementioned rules are given in Table I (Rule 1: without normalization) and Table II (Rule 2: normalized results). For one of the patients, the conductivity could not be determined.

TABLE I

| | | Negative | | Positive | | All | |
|---|---|---|---|---|---|---|---|
| | | N | Column % | N | Column % | N | Column % |
| Outcome on | Outcome: 72 h/30 d | | | | | | |
| Admission | | | | | | | |
| Hospit. | Any Cardiac | | | 10 | (100.0%) | 10 | (100.0%) |
| CCU/Cardiol. | Event | | | | | | |
| | Free of Chest | | | 1 | (100.0%) | 1 | (100.0%) |
| | Pain | | | | | | |
| | All | | | 11 | (100.0%) | 11 | (100.0%) |
| Hospital Internal | Outcome: 72 h/30 d | | | | | | |
| Med. | Any Cardiac | 9 | (29.0%) | 22 | (70.9%) | 31 | (100.0%) |
| | Event | | | | | | |
| | Free of Chest | | | 10 | (100.0%) | 10 | (100.0%) |
| | Pain | | | | | | |
| | All | 9 | (21.9%) | 32 | (78.0%) | 41 | (100.0%) |
| Discharged | Outcome: 72 h/30 d | | | | | | |
| | Any Cardiac | 2 | (12.5%) | 14 | (87.5%) | 16 | (100.0%) |
| | Event | | | | | | |
| | Free of Chest | 6 | (28.5%) | 15 | (71.4%) | 21 | (100.0%) |
| | Pain | | | | | | |
| | All | 8 | (21.6%) | 29 | (78.3%) | 37 | (100.0%) |
| All | Outcome: 72 h/30 d | | | | | | |
| | Any Cardiac | 11 | (19.2%) | 46 | (80.7%) | 57 | (100.0%) |
| | Event | | | | | | |
| | Free of Chest | 6 | (18.7%) | 26 | (81.2%) | 32 | (100.0%) |
| | Pain | | | | | | |
| | All | 17 | (19.1%) | 72 | (80.8%) | 89 | (100.0%) |

TABLE II

| | | Negative | | Positive | | All | |
|---|---|---|---|---|---|---|---|
| | | N | Column % | N | Column % | N | Column % |
| Outcome on | Outcome: 72 h/30 d | | | | | | |
| Admission | | | | | | | |
| Hospital | Any Cardiac | 1 | (10.0%) | 9 | (90.0%) | 10 | (100.0%) |
| CCU/Cardiology | Event | | | | | | |
| | Free of Chest | 1 | (100.0%) | | | 1 | (100.0%) |
| | Pain | | | | | | |
| | All | 2 | (18.1%) | 9 | (81.8%) | 11 | (100.0%) |
| Hospital Internal | Outcome: 72 h/30 d | | | | | | |
| Medicine | Any Cardiac | 12 | (40.0%) | 18 | (60.0%) | 30 | (100.0%) |
| | Event | | | | | | |
| | Free of Chest | 4 | (40.0%) | 6 | (60.0%) | 10 | (100.0%) |
| | Pain | | | | | | |
| | All | 16 | (40.0%) | 24 | (60.0%) | 40 | (100.0%) |
| Discharged | Outcome: 72 h/30 d | | | | | | |
| | Any Cardiac | 3 | (18.7%) | 13 | (81.2%) | 16 | (100.0%) |
| | Event | | | | | | |
| | Free of Chest | 9 | (42.8%) | 12 | (57.1%) | 21 | (100.0%) |
| | Pain | | | | | | |
| | All | 12 | (32.4%) | 25 | (67.5%) | 37 | (100.0%) |
| All | Outcome: 72 h/30 d | | | | | | |
| | Any Cardiac | 16 | (28.5%) | 40 | (71.4%) | 56 | (100.0%) |
| | Event | | | | | | |
| | Free of Chest | 14 | (43.7%) | 18 | (56.2%) | 32 | (100.0%) |
| | Pain | | | | | | |
| | All | 30 | (34.0%) | 58 | (65.9%) | 88 | (100.0%) |

The sensitivity and specificity of each of the two Rules (i.e. non-normalized and conductivity-normalized thromboxane measurements) for each of the patient groups are shown in Table III (non-normalized data) and Table IV (conductivity-normalized data).

TABLE III

Non-normalized Thromboxane Data

| Patient Group | Sensitivity % | Specificity % |
|---|---|---|
| Hospital | 100 | 0 |
| CCU/Cardiology | | |
| Hospital Internal | 70.9 | 0 |
| Medicine | | |
| Discharged | 87.5 | 28.5 |
| All | 80.7 | 18.7 |

TABLE IV

Conductivity-normalized Thromboxane Data

| Patient Group | Sensitivity % | Specificity % |
|---|---|---|
| Hospital | 90 | 100 |
| CCU/Cardiology | | |
| Hospital Internal | 60 | 40 |
| Medicine | | |
| Discharged | 81.2 | 42.8 |
| All | 71.4 | 43.7 |

It may be seen from Tables III and IV that while both interpretive rules yield results having approximately similar sensitivity levels, Rule 2 (that uses conductivity-normalized thromboxane concentrations) gives much improved specificity results. The significance of the higher specificity level of the conductivity-normalized method is that it may be used to determine in a highly reliable manner which of the patients have not suffered from an acute cardiac episode, and therefore may be discharged. It is thus concluded that the conductivity-normalized thromboxane B₂ concentration data provides a much more reliable tool for predictive use in the diagnosis of acute coronary conditions.

While specific embodiments of the invention have been described for the purpose of illustration, it will be understood that the invention may be carried out in practice by skilled persons with many modifications, variations and adaptations, without departing from its spirit or exceeding the scope of the claims.

1. A method for the diagnosis of a medical condition in a subject, comprising the steps of:
a) obtaining a sample of urine from s. For one of the patients, the conductivity could not be determined.aid subject;
b) measuring the concentration of one or more analytes relevant for the diagnosis of said medical condition in said urine sample;
c) measuring the electrical conductivity of said urine sample;
d) obtaining a normalized value (NTV) for the analyte concentration by dividing said analyte concentration obtained in step b) by the electrical conductivity of the urine sample obtained in step c);
e) determining whether said subject is suffering from said medical condition by means of comparing the NTV obtained in step d) with a pre-determined reference value.

2. A method according to claim 1, wherein the medical condition to be diagnosed is an acute cardiac condition, and wherein the relevant urinary analyte comprises one or more thromboxanes selected from the group consisting of thromboxane B₂, 11-dehydrothromboxane B₂, 2,3-di-northromboxane B₂, and mixtures thereof,

3. A method according to claim 2, wherein the thromboxane measured is thromboxane B₂.

4. A method according to claim 2, wherein both the thromboxane concentration and the electrical conductivity are measured using an amperometric assay.

5. A method according to claim 2, wherein both the thromboxane concentration and the electrical conductivity are measured using a semiconductor-based device.

6. A method according to claim 2, wherein the electrical conductivity is measured using a conductivity meter.

7. A method according to claim 2, wherein the thromboxane concentration is determined using an immunoassay.

8. A method according to claim 7, wherein the immunoassay is an enzymeimmunoassay.
